(19) United States
(12) Patent Application Publication
(10) Pub. No.: US 2022/0338905 A1
(43) Pub. Date: Oct. 27, 2022

(54) METHODS AND SYSTEMS FOR CONSTRAINT OF SPINOUS PROCESSES WITH ATTACHMENT

(71) Applicants: The Board of Trustees of the Leland Stanford Junior University, Stanford, CA (US); Empirical Spine, Inc., Woodside, CA (US)

(72) Inventors: Todd ALAMIN, Woodside, CA (US); Ian BENNETT, San Francisco, CA (US); Louis FIELDING, Portland, OR (US); Colin CAHILL, San Francisco, CA (US)

(21) Appl. No.: 17/541,164

(22) Filed: Dec. 2, 2021

Related U.S. Application Data
(63) Continuation of application No. 16/140,147, filed on Sep. 24, 2018, now abandoned.
Application No. 16/140,147 is a continuation of application No. 15/409,436, filed on Jan. 18, 2017, now Pat. No. 10,080,589.
Application No. 15/409,436 is a continuation of application No. 14/875,495, filed on Oct. 5, 2015, now abandoned.
Application No. 14/875,495 is a continuation of application No. 13/958,323, filed on Aug. 2, 2013, now Pat. No. 9,149,304.
Application No. 13/958,323 is a continuation of application No. 11/827,980, filed on Jul. 13, 2007, now Pat. No. 8,523,904.
Application No. 11/827,980 is a continuation-in-part of application No. 11/076,469, filed on Mar. 9, 2005, now Pat. No. 7,458,981.
(60) Provisional application No. 60/862,085, filed on Oct. 19, 2006.
(60) Provisional application No. 60/551,235, filed on Mar. 9, 2004.

(51) Int. Cl. A61B 17/70; A61B 17/84

(52) U.S. Cl. CPC A61B 17/7067; A61B 17/7055; A61B 17/842; A61B 17/7062; A61B 17/7053; A61B 17/846; A61B 17/683

(57) ABSTRACT

Spinal implants for limiting flexion of the spine are implanted between a superior spinous process and an inferior spinous process or sacrum. The implants include upper straps which are placed over the upper spinous process, while the lower portions of the implant are attached to the adjacent vertebra or sacrum. The attachments may be fixed, for example using screws or other anchors, or may be non-fixed, for example by placing a loop strap through a hole in the spinous process or sacrum.

### CROSS REFERENCE TO RELATED APPLICATIONS

This application is a continuation of U.S. patent application Ser. No. 16/140,147, filed on Sep. 24, 2018, now U.S. Patent Application Publication No. 2019/0262043, which is a continuation of U.S. patent application Ser. No. 15/409,436, filed Jan. 18, 2017, now U.S. Pat. No. 10,080,589, which is a continuation of U.S. patent application Ser. No. 14/875,495, filed on Oct. 5, 2015, Publication No. US-2016-0256201-A1, which is a continuation of U.S. patent application Ser. No. 13/958,323, filed on Aug. 2, 2013, now U.S. Pat. No. 9,149,304, which is a continuation of U.S. patent application Ser. No. 11/827,980, filed on Jul. 13, 2007, now U.S. Pat. No. 8,523,904, which claims the benefit of U.S. Provisional Patent Application No. 60/862,085, filed on Oct. 19, 2006, each of which is herein incorporated by reference in its entirety.

U.S. patent application Ser. No. 11/827,980 is also a continuation-in-part of U.S. patent application Ser. No. 11/076,469, filed on Mar. 9, 2005, now U.S. Pat. No. 7,458,981, which claimed the benefit of U.S. Provisional Patent Application No. 60/551,235, filed on Mar. 9, 2004, each of which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

### 1. Field of the Invention

The present invention relates generally to medical methods and apparatus. More particularly, the present invention relates to methods and devices for restricting spinal flexion in patients having back pain or other spinal conditions.

A major source of chronic low back pain is discogenic pain, also known as internal disc disruption. Patients suffering from discogenic pain tend to be young, otherwise healthy individuals who present with pain localized to the back. Discogenic pain usually occurs at the discs located at the L4-L5 or L5-S1 junctions of the spine (FIG. 1). Pain tends to be exacerbated when patients put their lumbar spines into flexion (i.e. by sitting or bending forward) and relieved when they put their lumbar spines into extension (i.e. arching backwards). Discogenic pain can be quite disabling, and for some patients, can dramatically affect their ability to work and otherwise enjoy their lives.

This pain experienced by patients with discogenic low back pain can be thought of as flexion instability, and is related to flexion instability that is manifested in other conditions. The most prevalent of these is spondylolisthesis, a spinal condition in which abnormal segmental translation is exacerbated by segmental flexion. The device described here should as such also be useful for these other spinal disorders associated with segmental flexion, for which the prevention or control of spinal segmental flexion is desired.

Current treatment alternatives for patients diagnosed with chronic discogenic pain are quite limited. Many patients follow a conservative treatment path, such as physical therapy, massage, anti-inflammatory and analgesic medications, muscle relaxants, and epidural steroid injections, but typically continue to suffer with a significant degree of pain. Other patients elect to undergo spinal fusion surgery, which commonly requires discectomy (removal of the disk) together with fusion of adjacent vertebra. Fusion is not usually recommended for discogenic pain because it is irreversible, costly, associated with high morbidity, and of questionable effectiveness. Despite its drawbacks, however, spinal fusion for discogenic pain remains common due to the lack of viable alternatives.

Recently, a less invasive and potentially more effective treatment for discogenic pain has been proposed. A spinal implant has been designed which inhibits spinal flexion while allowing substantially unrestricted spinal extension. The implant is placed over one or more adjacent pairs of spinal processes and provides an elastic restraint to the spreading apart of the spinal processes which occurs during flexion. Such devices and methods for their use are described in U.S. Patent Application Publication No. 2005/02161017A1, published on Sep. 29, 2005, and having common inventors with the present application.

As illustrated in FIG. 2, an implant 10 as described in the '017 application, typically comprises an upper strap component 12 and a lower strap component 14 joined by a pair of compliant members 16. The upper strap 12 is shown disposed over the top of the spinous process SP4 of L4 while the lower strap 14 is shown extending over the bottom of the spinous process SP5 of L5. The compliance member 16 will typically include an internal element, such as a spring of rubber block, which is attached to the straps 12 and 14 in such a way that the straps may be “elastically” or “compliantly” pulled apart as the spinous processes SP4 and SP5 move apart during flexion. In this way, the implant provides an elastic tension on the spinal processes which provides a force that resists flexion. The force increases, typically linearly with a non-variable spring constant, as the processes move further apart. Usually, the straps themselves will be essentially non-compliant so that the degree of elasticity or compliance may be controlled and provided solely by the compliance members 16.

Although providing significant benefits, the system illustrated in FIG. 2 can be difficult to implant in certain patient anatomies where the spinous processes are relatively small or have certain geometries. Moreover, the systems are not intended for implantation at the L5-S1 junction as the spinous process on the sacrum is not always sufficient for attachment with this system.

For these reasons, it would be desirable to provide improved spinal implants and methods for their use for inhibiting flexion in patients suffering from discogenic pain. It would be particularly desirable if the improved implants and methods would be suitable for implantation at the L5-S1 junction and in patients having anatomies which prevent other difficulties for implantation of the prior systems as described in the '017 application. At least some of these objectives will be met by the inventions described hereinbelow.

### 2. Description of the Background Art

US 2005/0216017A1 has been described above. Other patents and published applications of interest include: U.S. Pat. Nos. 4,966,600; 5,011,494; 5,092,866; 5,116,340; 5,282,863; 5,395,374; 5,415,658; 5,415,661; 5,449,361; 5,456,722; 5,462,542; 5,496,318; 5,540,698; 5,609,634; 5,645,599; 5,725,582; 5,902,305; Re. 36,221; 5,928,232; 5,935,133; 5,964,769; 5,989,256; 6,053,921; 6,312,431; 6,364,883; 6,378,289; 6,391,030; 6,468,309; 6,436,099; 6,451,019; 6,582,433; 6,605,091; 6,626,944; 6,629,975; 6,652,527; 6,652,585; 6,656,185; 6,669,729; 6,682,533; 6,689,140; 6,712,819; 6,689,168; 6,695,852; 6,716,245; 6,761,720; 6,835,205; Published U.S. patent application Nos. US 2002/0151978; US 2004/0024458; US 2004/0106995; US 2004/0116927; US 2004/0117017; US 2004/0127989; US 2004/0172132; US 2005/0033435; US 2005/0049708; US 2006/0069447; Published PCT Application Nos. WO 01/28442 A1; WO 02/03882 A2; WO 02/051326 A1; WO 02/071960 A1; WO 03/045262 A1; WO 2004/052246 A1; WO 2004/073532 A1; and Published Foreign Application Nos. EP 0322334 A1; and FR 2 681 525 A1.

### BRIEF SUMMARY OF THE INVENTION

The present invention provides spinal implants and methods for restricting spinal flexion for the treatment of discogenic pain and other spinal conditions, such as spondylolisthesis, in which the physician may desire to control segmental flexion. The methods comprise positioning a first segment of a tether structure over a spinous process of a vertebra without attachment. At least one other segment of the tether structure is attached to an adjacent vertebra or sacrum, and at least a portion of the tether structure is adapted to elastically elongate to apply tension between the spinous process and the adjacent vertebra or sacrum as the spine undergoes flexion, i.e., as the spinous process moves apart from the adjacent vertebra or sacrum as the patient leans forward. The methods and implants of the present invention are particularly useful for treating the L4-L5 and the L5-S1 junctions of the spine (FIG. 1). The first segment of the tether structure is generally a loop similar or identical to strap 12 in FIG. 1 which is non-fixedly attached to a spinous process, typically being placed over a superior spinous process but not being otherwise attached to the spinous process. Thus, the first segment of the tether will be able to move or shift laterally and/or in the anterior-posterior direction relative to the spinous process as the spine undergoes flexion and extension.

The at least one other segment of the tether may be attached to the adjacent vertebra or sacrum in a variety of ways. In a first group of embodiments, the at least one other segment of the tether structure will be fixedly attached to the adjacent vertebra or sacrum so that the segment will not move relative to a point of attachment. For example, the other segment of the tether structure may comprise two separate end segments which are fixedly attached to the vertebra or sacrum, for example with screws, dowels, staples, pins, sutures, or the like. When attached to a vertebra, the two separate end segments may be attached to opposed sides of a spinous process on an inferior vertebra. When attached to a sacrum, the two separate end segments may be attached to an alar surface of the sacrum, typically with alar screws.

In a second set of embodiments, the at least one other segment of the tether structure may be non-fixedly attached to the adjacent vertebra or sacrum so that the segment can move or shift relative to a point of attachment. For example, the at least one other segment may comprise a loop similar to the lower strap 14 of FIG. 2. A hole may be formed in the spinous process of an adjacent vertebra so that the loop may be passed through the hole to provide a non-fixed attachment. Similarly, a hole could be formed in a protruding surface structure on the sacrum to receive the lower loop segment of the tether structure. Alternatively, such a loop segment could be passed through the eye(s) of one or more islet screws which are implanted into the lower vertebra or sacrum.

The tether structure will typically comprise at least one compliance member and more typically comprise two compliance members, generally as described in connection with the embodiment in FIG. 2. When the tether structure comprises at least two compliance members, there will be at least one loop segment or strap extending between the upper ends of the compliance members. The strap will usually be non-compliant but could in other embodiments have a limited compliance or flexibility. The tether structure may comprise a further lower loop segment or strap, generally as illustrated in FIG. 2, when the tether structure is intended to pass through an islet or hole in the lower vertebra or sacrum. Alternatively, the tether structure will comprise at least two additional segments having separate ends which extend from each of the two compliance members. The separate ends will be adapted for anchoring to the adjacent vertebra or sacrum using screws, dowels, staples, or any of the techniques described above.

In all cases, the tether structure will typically provide little or no restriction or resistance to extension of the spine. Most often, the tether structure will be free from components or other structures which are located between the adjacent spinous processes or between the spinous processes and the adjacent sacrum. In other instances, however, a cross-member or other low profile structure may be placed between the two compliance members to maintain alliance of the compliance members, generally as described in co-pending application Ser. No. 11/777,366, filed on the same day as the present application. The use of cross-members for stabilizing the compliance members may be advantageous when the lower portion of the tether structure is non-fixedly attached to the lower vertebra or sacrum.

In a further aspect of the present invention, a spinal implant comprises at least two compliance members, where each compliance member has an upper and a lower end. An upper tether structure extends between the upper ends of the two compliance members and is adapted for placement over a spinous process of a first vertebra. Typically, the upper tether structure will be a non-compliant strap. The spinal implant further comprises a first lower tether structure attached at an upper end to the lower end of the compliance member and having a lower end adapted to be fixedly attached to a vertebra or sacrum adjacent to the first vertebra. A second lower tether segment is attached at its upper end to a lower end of the second compliance member and has a lower end adapted to be fixedly attached to the vertebra or sacrum adjacent to the first vertebra. The lower ends of the first and second lower tether segments are typically non-compliant straps and may be adapted to be screwed into the adjacent vertebra or sacrum. Alternatively, the lower ends of the first and second lower tether segments may be adapted to be attached to a dowel implanted in the adjacent vertebra or sacrum. The spinal implant may optionally comply to screws, anchors, or other attachment members for fixedly attaching the lower ends of the tether segments to the vertebra or sacrum.

### BRIEF DESCRIPTION OF THE DRAWINGS

FIG. 1 is a schematic diagram illustrating the lumbar region of the spine including the spinous processes (SP), facet joints (FJ), lamina (L), transverse processes (TP), and sacrum (S).

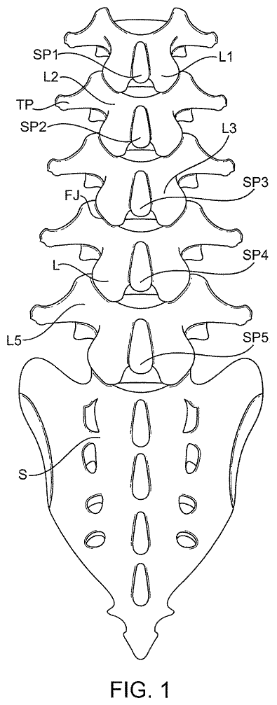

FIG. 2 illustrates a spinal implant of the type described in US 2005/0216017A1.

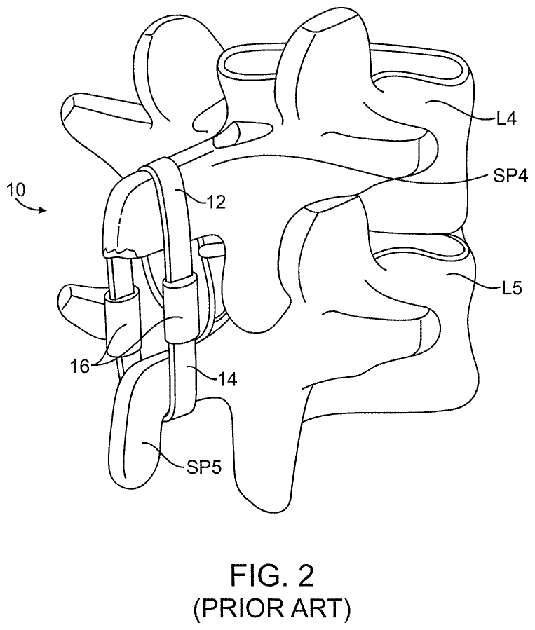

FIG. 3 illustrates a first embodiment of a spinal implant adapted to be placed between a pair of spinous processes and having a lower tether segment non-fixedly attached to the lower spinous process.

FIG. 4 is a second embodiment of a spinal implant adapted to be placed between adjacent spinous processes and having a lower segment adapted to be fixedly attached to the lower spinous process.

FIG. 5 illustrates a third embodiment of a spinal implant according to the present invention having an upper end placed over the spinous process of L5 and a lower end non-fixedly attached to the sacrum.

FIG. 6 illustrates a fourth embodiment of a spinal implant according to the present invention having an upper end secured over a spinous process of L5 and two separate lower segments attached to a dowel implanted in the sacrum.

FIG. 7 illustrates a fifth embodiment of a spinal implant according to the present invention having an upper segment placed over a spinous process of L5 and two separate lower segments fixedly attached by alar screws to the sacrum.

FIG. 8 illustrates a sixth embodiment of a spinal implant according to the present invention having an upper segment placed over a spinous process of L5 and two separate lower segments fixedly attached by superior articular facet screws to the sacrum.

FIG. 9 illustrates a seventh embodiment of a spinal implant according to the present invention having an upper segment placed over a spinous process of L5 and two separate lower tether segments each of which passes through a hole created in the superior articular facet of S1 and is non-fixedly attached via a toggle anchor (t-anchor).

FIG. 10 illustrates an eighth embodiment of a spinal implant according to the present invention having an upper segment placed over a spinous process of L5 and two separate lower tether segments each of which is connected to a hook attached to the dorsal S1 foramen.

### DETAILED DESCRIPTION OF THE INVENTION

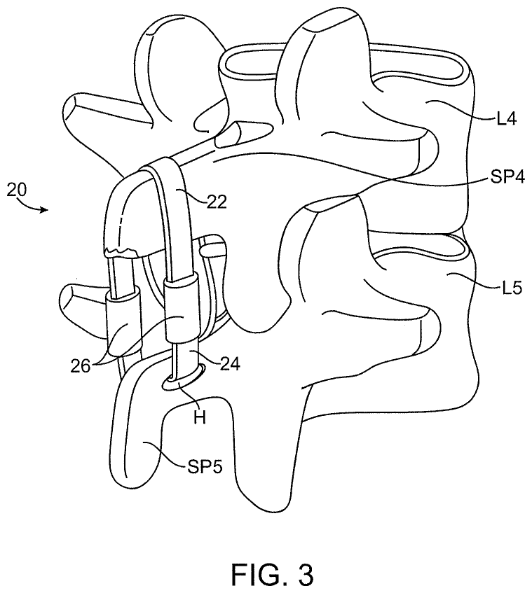

Referring now to FIG. 3, a spinal implant 20 suitable for use in accordance with the methods of the present invention comprises an upper strap 22, a lower strap 24, and a pair of compliance members 26 joining the upper and lower straps. Typically, the upper and lower straps 22 and 24 will be non-distensible but will be joined to the compliance members 26 so that they can be expanded from a constricted configuration, as shown in broken line, when the patient's spine is in a neutral position between flexion and extension, to an expanded configuration (shown in full line) when the patient's spine is in flexion. The compliance members 26 will provide a force which acts against the extension of the spinous processes SP4 and SP5, as generally described in prior patent application U.S. 2005/0216017, which has been previously incorporated herein by reference. In contrast to the teachings of the '017 application, however, the lower strap 24 is non-fixedly attached to the spinous process SP5 of L5. By passing through a hole H formed in the spinous process SP5, the lower strap 24 is maintained stably and will not be displaced.

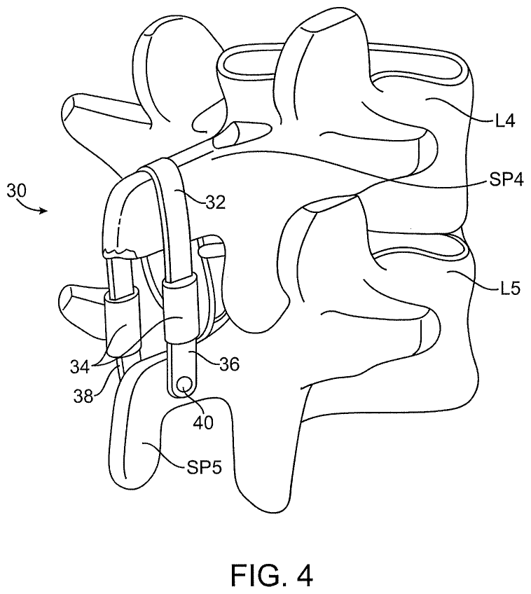

Referring now to FIG. 4, a spinal implant 30 may comprise a tether structure including an upper strap 32, a pair of compliance members 34, and first and second lower straps 36 and 38, one strap extending from each of the compliance members 34. The lower straps 36 will typically be non-compliant, as is the upper strap 32, with the compliance and elasticity being provided by compliance members 34. The lower ends of the lower straps 36 and 38 may be fixedly attached to the spinous process SP5 using screws 40 or any other suitable anchors. By using the screw or other anchors, the lower straps 36 and 38 will be fixedly attached to the spinous process SP5, permitting no relative movement between the straps 36 and 38 and the spinous process SP5 and L5. The upper strap 32, in contrast, will be able to move or shift slightly relative to the upper spinous process SP4 on L4, although the interspinous ligament that stretches between L4 and L5 (through which the strap passes) will resist motion in the anterior-posterior direction.

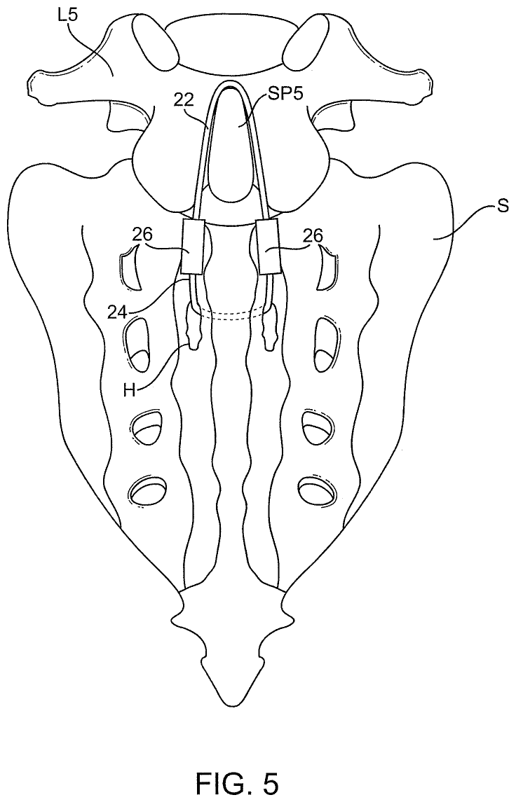

Referring now to FIG. 5, the spinal implant 20, generally described in FIG. 3, may also be implanted between the spinous process SP5 of L5 and the sacrum S. The upper strap 22 will be placed over spinous process SP5 while the lower strap 24 will be placed through a hole H placed in a surface ridge on the dorsal surface of the sacrum.

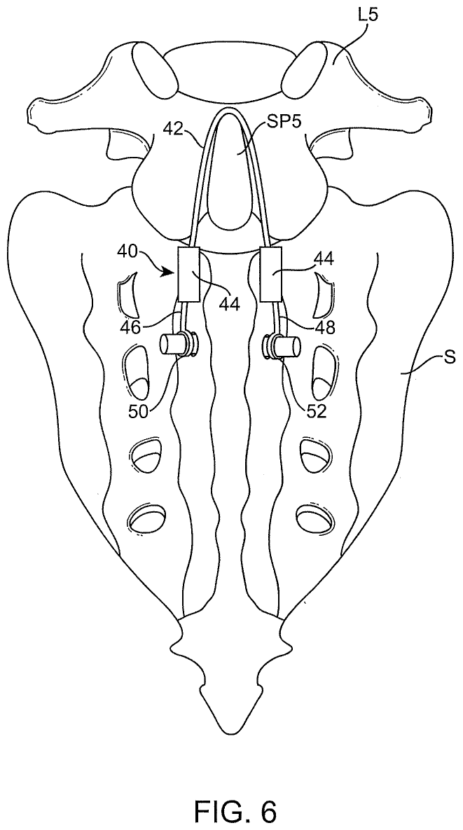

Referring now to FIG. 6, a spinal implant 40 comprising an upper strap 42, a pair of compliance members 44 and lower strap segments 46 and 48 may be implanted over the spinous process SP5 of L5 and the sacrum S. In particular, a dowel or other anchor element may be implanted in the S1 spinous process of the sacrum (which is typically small relative to the L5 spinous process and less able to provide an anchor around which a strap can be looped) and rings 50 and 52 at the lower ends of the lower strap segments 46 and 48 may be placed over the dowel or other anchor.

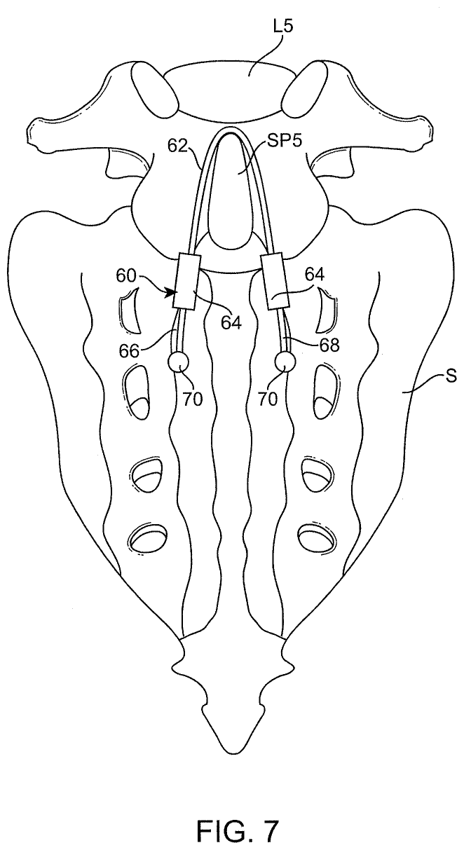

As illustrated in FIG. 7, a further alternative for implanting an implant 60 is illustrated. Implant 60 comprises an upper strap 62, a pair of compliance members 64 and lower strap segments 66 and 68. The upper strap segment is placed over spinous process SP5 of L5 while the lower strap segments 66 and 68 are anchored on the alar region of the sacrum by alar screws 70.

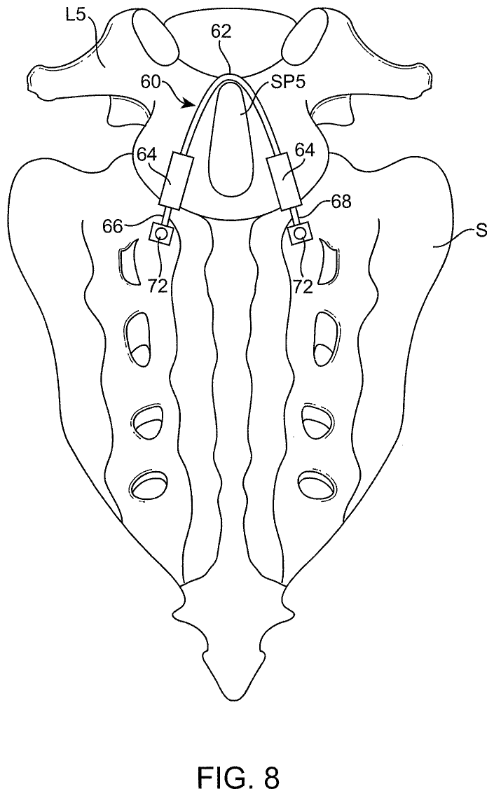

As illustrated in FIG. 8, a further alternative for implanting an implant 60 is illustrated. Implant 60 comprises an upper strap 62, a pair of compliance members 64 and lower strap segments 66 and 68. The upper strap segment is placed over spinous process SP5 of L5 while the lower strap segments 66 and 68 are anchored to superior articular facets of the sacrum by superior articular facet screws 72.

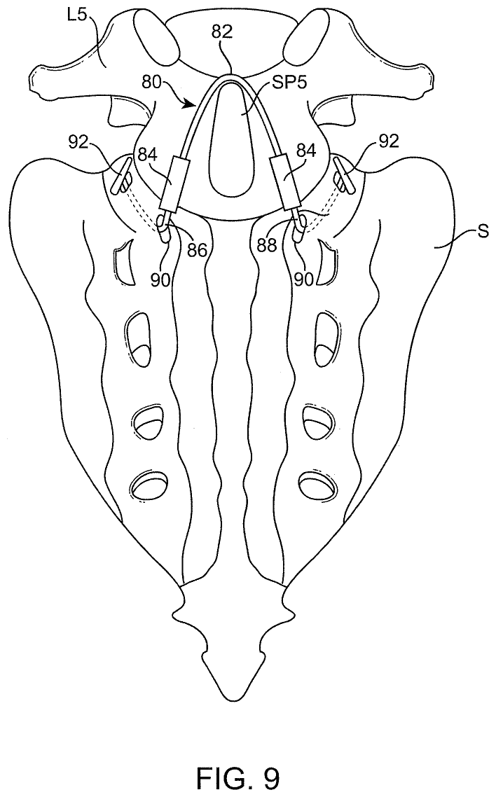

As illustrated in FIG. 9, a further alternative for implanting an implant 80 is illustrated. Implant 80 comprises an upper strap 82, a pair of compliance members 84 and lower strap segments 86 and 88. The upper strap segment is placed over spinous process SP5 of L5 while the lower strap segments 86 and 88 pass dorsal-medial to proximal-lateral through holes 90 created in the superior articular facet of S1 and are non-fixedly attached via toggle anchors (t-anchors) 92 on the proximal-lateral side of the facets.

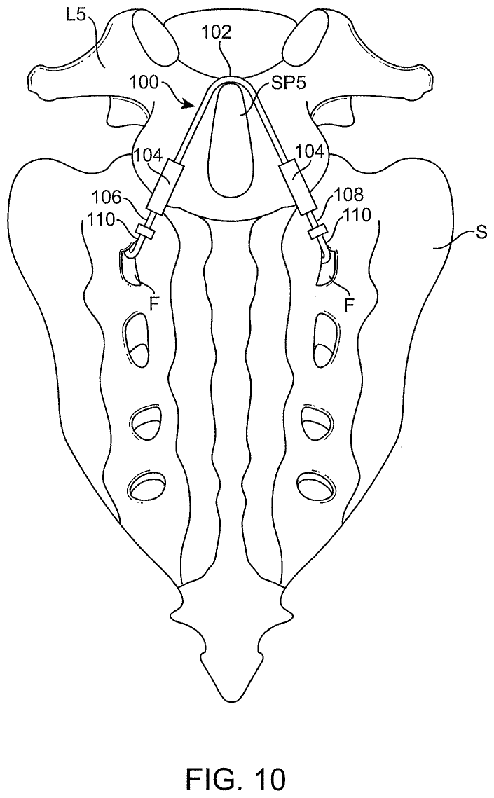

As illustrated in FIG. 10, a further alternative for implanting an implant 100 is illustrated. Implant 100 comprises an upper strap 102, a pair of compliance members 104 and lower strap segments 106 and 108. The upper strap segment is placed over spinous process SP5 of L5 while the lower strap segments 106 and 108 are connected to hooks 110 attached to the dorsal S1 foramen F.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for restricting flexion of a spine, said method comprising:
non-fixedly attaching one segment of a tether structure over a superior spinous process of a vertebra and at least one other segment of the tether structure to a sacrum;
wherein the tether structure comprises at least one compliance member which elastically elongates to controllably restrict flexion.
